(19) United States
(12) Patent Application Publication
(10) Pub. No.: US 2004/0052750 A1
(43) Pub. Date: Mar. 18, 2004

(54) Compositions for prevention and alleviation of skin wrinkles

(76) Inventors: Hak-Mo Lee, Daejeon (KR); Sang-Nyun Kim, Daejeon (KR); Moon-Moo Kim, Daejeon (KR); Ho-Jeong Ahn, Daejeon (KR); Kyung-Ok No, Chungbuk (KR)

Correspondence Address: Albert Wai Kit Chan, Law Offices of Albert Wai Kit Chan, World Plaza Suite 604, 141 07 20th Avenue, Whitestone, NY, 11357, US

(21) Appl. No.: 10/250,596

(22) PCT Filed: Dec. 19, 2001
(86) PCT No.: PCT/KR01/02208

(30) Foreign Application Priority Data
Dec. 20, 2000 (KR) 2000/78966

(51) Int. Cl. A61K 38/13; A61K 7/06; A61K 7/11

(52) U.S. Cl. 424/70.14; 514/11

(57) ABSTRACT

The present invention discloses a topical composition for prevention and alleviation of wrinkling which comprises one or two or more selected from the group consisting of Phenytoin, Valproic acid, Cyclosporin A, Nifedipine, Diltiazem, Verapamil HCl and Amoldipine as an active ingredient having an effect of boosting collagen synthesis.

### TECHNICAL FIELD

The present invention relates to a topical composition for prevention and alleviation of skin wrinkles which comprises one or two or more selected from the group consisting of phenytoin, valproic acid, cyclosporine A, nifedipine, diltiazem, verapamil HCl and amoldipine as an active ingredient having an effect of promoting collagen synthesis, in conjunction with conventional components of a formulation for transdermal absorption such as cream, ointment, lotion, skin tonic, gel, pack, patch or patch-type administering apparatus.

### BACKGROUND ART

Skin aging is developed by both endogenous causes, for example, aging, and environmental causes. The effects of aging are shown as wrinkles in the skin, which include neck wrinkles, worry lines, frown lines, crow's feet, the folds from the side of the nose to the corners of the mouth, and fine lines around the eyes, below the lips, and over the face. Skin wrinkles caused by aging, though there are individual differences, commonly occur in individuals in their early twenties and increase with age. With aging, the amount of dermal collagen of skin is decreased and alterations in elastic fibers occur, whereby the skin relaxes and fine wrinkles appear. Meanwhile, collagen is a major matrix protein produced by fibroblasts of the skin, being present in the extracellular matrix. It is a primary protein comprising 30% by weight of proteins in the human body, and has a firm structure of a triple helix. It is known that collagen functions to provide structural stability to the skin, durability of connective tissues and cohesion of tissues while supporting cell coherence, cell proliferation, and induction of differentiation of unspecialized cells. Also, it is known that collagen is broken down by exposure to UV, an environmental cause of skin aging, and the damage by UV is proportional to the accumulated time of exposure thereto. UV denatures collagenous fibers, causing wrinkles and decreasing elasticity of the skin. Other environmental causes known to promote skin aging include wind, heat and smoking.

As mentioned above, collagen is closely related with skin aging. The amount of collagen in the dermis is decreased with aging and by UV radiation. Collagen decreases by 65% from age 20 to age 80. Such a decrease of collagen makes the skin thin and further, is closely associated with the formation of skin wrinkles.

Studies have been widely performed to find a method for the prevention and alleviation of skin wrinkles, elucidating important roles of collagen. The studies also elucidated that when collagen synthesis is activated in skin, dermal matrix components are increased, which has effects including alleviation of wrinkles, and increased elasticity and strength of skin. Therefore, using collagen having a moisture retention effect, some collagen-incorporated cosmetics have been developed. Such cosmetics, however, are poor in holding moisture, since the cosmetics are applied to the surface of skin and high molecular weight collagen is poor intransdermal absorption. As a result, their use fails to provide an intrinsic improvement in skin appearance. In the prior art, retinoic acid, TGF-β, protein derived from an animal placenta (JP8-231370), betulinic acid (JP8-208424) and Chlorella extract (JP9-40523, JP10-36283) are disclosed as substances for promoting collagen synthesis. As for retinoic acid, it is unstable and has a problem in its safety due to causing irritation and redness upon application the skin, limiting the available dosage thereof. As for other above substances including Chlorella extract, their effects of increasing collagen synthesis are weak, so they hardly improve skin appearance. Recently, several new procedures for treating wrinkles by promoting collagen synthesis have been introduced. Examples include ultrasonic treatment, skin scaling, laser peeling, botulinum toxin injection and Restilene injection. These procedures, however, have disadvantages in terms of cost effectiveness and duration of their effects. Thus, it is desirable to search for and develop a highly effective agent for promoting collagen synthesis.

### DISCLOSURE OF THE INVENTION

Therefore, the present inventors have conducted studies to develop a compound having an effect of promoting collagen synthesis, and found that phenytoin, valproic acid, cyclosporine A, nifedipine, diltiazem, verapamil HCl and amoldipine which are already known as anticonvulsants, immunosuppressants or calcium channel inhibitors have yery strong effects of promoting collagen synthesis in human fibroblast cell lines. Further, it was found that as applied to the skins of rats and mice, the compounds exhibited strong inhibition and alleviation effects of wrinkles, proving the effects of inhibiting and preventing signs of skin aging such as skin wrinkles. Accordingly, the present invention is directed to a composition comprising phenytoin, valproic acid, cyclosporine A, nifedipine, diltiazem, verapamil HCl or amoldipine as an active ingredient having an effect of promoting collagen synthesis.

Phenytoin and valproic acid have been widely used as anticonvulsants for treatment of epilepsy, and their effects on collagen synthesis are documented (U.S. Pat. No. 5686489; Minerva Stomatol., 47(9): 3 87-398, September 1998). Cyclosporine A has been widely used as an immunosuppressant for suppressing rejection of tissues after transplantation, and its effects on collagen synthesis are reported (J Periodontol., 72(7): 921-931, July 2001). Nifedipine, diltiazem, verapamil HCl and amoldipine have already been used as calcium channel inhibitors, and their effects on collagen synthesis are also reported (J Periodontol., 72(8), August 2001; Proc Natl Acad Sci USA, 93(11): 5478-5482, May 1996; J Urol., 156(6): 2 067-2072, December 1996). However, the above drugs are not disclosed for use as topical agents applied to the skin for preventing and alleviating skin wrinkles, as in the present invention.

Hereinafter, a topical composition for preventing and alleviating skin wrinkles will be described in detail, in conjunction with experimental examples and examples.

### EXPERIMENTAL EXAMPLE 1

### Effect of Active Ingredients of the Invention on Promoting Collagen Production in Fibroblasts

To investigate the effects of active ingredients of the invention on promoting collagen production in fibroblasts in cellular level, respective active ingredients were added to cultures of fibroblasts derived from a human. The synthesized collagen was measured using a modification of a method proposed by Martens (Gut, 33: 1664-1670, 1992) to evaluate the effects of the active ingredients. The experimental protocol in detail is as follows.

Human-derived fibroblasts were transferred to a 24 well plate and cultured in a medium containing 10% fetal bovine serum (FBS) for 24 hours, followed by washing twice with phosphate buffered saline. The cells were then incubated in a medium containing 1% FBS in the presence of phenytoin, valproic acid, cyclosporinee A, nifedipine, diltiazem, verapamil HCl or amoldipine at final concentrations of 10-8 to 10-5 M. After 1 hr incubation, cultures were added with 10 μCi of ³H-proline per well, followed by a final incubation for 24 hours. After the incubation was terminated, cells from each group were harvested and two fractions of each culture were prepared. One fraction from each culture was treated with collagenase. To all fractions was added trichloroacetic acid to precipitate proteins. The amount of radioactivity incorporated into collagenase-sensitive protein was measured and compared with that of the other fraction which was not treated with collagenase. The difference in radioactivity was attributed to the promoting effect of the compound. Samples without an active ingredient served as a control group, the amount of collagen synthesized being 100%. The results are shown in Table 1.

TABLE 1
Effect of promoting collagen production in fibroblasts (%)

| | | Exp. 1 | Exp. 2 | Exp. 3 | Exp. 4 |
|---|---|---|---|---|---|
| Compound/Conc. | Control 1 0 M | 10⁻⁸ M | 10⁻⁷ M | 10⁻⁶ M | 10⁻⁵ M |
| Phenytoin | 100.00 | 215.28* | 298.35 | 360.65 | 381.54 |
| Valproic acid | 100.00 | 201.13 | 283.24 | 332.11 | 370.21 |
| Cyclosporine A | 100.00 | 212.11 | 293.21 | 352.31 | 372.27 |
| Nifedipine | 100.00 | 204.31 | 292.21 | 330.30 | 358.16 |
| Diltiazem | 100.00 | 199.15 | 276.25 | 321.23 | 362.12 |
| Verapamil HCl | 100.00 | 183.25 | 280.23 | 331.09 | 355.12 |
| Amoldipine | 100.00 | 182.42 | 280.07 | 330.42 | 355.26 |

*Rate of collagen production = (collagen production of experimental group/collagen production of control group) × 100

As shown in Table 1, the active ingredients in experimental groups have effects of promoting collagen production with increasing concentration of the compounds, ranging from the minimum of 182.42% to the maximum of 381.54% in a dose-dependent manner, compared to the control group which contains no active ingredient of the invention. This demonstrates that the active ingredients of the invention have excellent effects on promoting collagen synthesis.

### EXPERIMENTAL EXAMPLE 2

### Promotion of Collagen Production in Rat Skin

The effects of application of active ingredients of the invention, that is, phenytoin, valproic acid, cyclosporinee A, nifedipine, diltiazem, verapamil HCl and amoldipine, on promoting collagen production in animal skin were investigated. The synthesized collagen was measured using a modification of a method proposed by Mard L DaCosta et al. (Surgery, 123: 287-293, 1998).

In brief, 5-week male SD rats were grouped with 5 rats per group. The rats were each incised 1 cm in the center of their abdomens and PVA sponges (Unipoint ind.) were inserted therein. After suturing, as for experimental groups, respective active ingredients to be examined were applied to the PVA sponge-embeded regions i n a volume o f 200 μl every day for 10 days. Upon autopsy, the PVA sponge was removed to quantify hydroxyproline. The PVA sponge was added with 4 ml of 6 N HCl, hydrolized at 130° C. for 3 hours and was subjected to complete drying. 50 μl of methanol was added and the solution was incubated at 110° C. until HCl was removed. 1.2 ml of 50% isopropanol was added to dissolve the remaining precipitate. 200 μl of chloramine-T (sodium p-toluensulfochloramide trihydrate) solution was added while stirring, and let stand for 10 min. After adding 1.2 ml of Ehrlich reagent and mixing, the solution was incubated at 50° C. for 90 min. The resulting solution was cooled to room temperature and absorbance at 558 nm was measured. Hydroxyproline standard solutions were prepared by dissolving 1 mg hydroxyproline in 1 ml HCl and diluting it to concentrations of 0, 0.2, 0.4, 0.8, 1 mg each relative to 25 μl of 6 N HCl. The standard solutions were hydrolyzed at 130° C. for 3 hours. The quantified value of hydroxyproline, relative to hydroxyproline value (100%) of the control group which was applied with solvent only, are shown in Table 2.

TABLE 2
Effect of promoting collagen production in animal skin (%)

| | | Exp. 1 | Exp. 2 | Exp. 3 | Exp. 4 |
|---|---|---|---|---|---|
| Compound/Conc. | Control 1 0 M | 10⁻⁸ M | 10⁻⁷ M | 10⁻⁶ M | 10⁻⁵ M |
| Phenytoin | 100.00 | 132.58* | 143.51 | 167.41 | 182.47 |
| Valproic acid | 100.00 | 128.05 | 139.24 | 157.72 | 178.13 |
| Cyclosporine A | 100.00 | 131.02 | 143.07 | 164.82 | 179.26 |
| Nifedipine | 100.00 | 129.92 | 142.41 | 161.43 | 185.88 |
| Diltiazem | 100.00 | 122.44 | 136.76 | 157.45 | 175.23 |
| Verapamil HCl | 100.00 | 135.63 | 147.39 | 167.06 | 183.32 |
| Amoldipine | 100.00 | 132.50 | 149.65 | 163.84 | 181.12 |

*Rate of collagen production = (hydroxyproline value of experimental group/hydroxyproline value of control group) × 100

As shown in Table 2, the active ingredients increased collagen production in rat skin and the rates of increase ranged from the minimum of 122.44% to the maximum of 185.88%, compared to the control group to which no active ingredient of the invention was applied. This demonstrates that the active ingredients of the invention strongly promote dermal collagen synthesis.

### EXPERIMENTAL EXAMPLE 3

### Effect on Inhibiting the Generation of Wrinkles in Hairless Mice

The effects of active ingredients of the invention, that is, phenytoin, valproic acid, cyclosporinee A, nifedipine, diltiazem, verapamil HCl and amoldipine, on inhibiting the generation of wrinkles in hairless mice were investigated.

6-week hairless mice were placed into 21 experimental groups and 3 control groups, with 10 rats per group. For experimental groups, mice were applied to the skin with respective compounds at a concentration of 10⁻⁸ to 10⁻³ M. The control groups were applied with solvent only, without any active ingredient. The experimental protocol in detail is as follows. Hairless mice were radiated using simulated sunlight at a dose of 2 MED (double Minimal Erythema Dose) 3 days a week for 12 weeks, thereby generating wrinkles. Respective active ingredients or the solvent only were applied twice every day (specifically, on radiation days, the application was performed at 30 min before and after the radiation), at a volume of 100 μl each for 10 weeks from the first radiation day. Degrees of inhibition of generated wrinkles were determined. The determination was performed by visual observation with naked eyes and photography. The degrees of inhibition of wrinkles in the compound-treatment groups (experimental groups) were compared with the control group (Score 0) and were determined as one of 4 stages, that is, none (Score 0), slight (Score 1), moderate (Score 2) and high (Score 3), and the corresponding mice were counted. The data are shown in Tables 3a to 3c.

TABLE 3a
Effect on inhibiting the generation of wrinkles in hairless mice

| | | Inhibition of wrinkles (number of mice) | | | |
|---|---|---|---|---|---|
| Group | Compound (10⁻⁸ M) | Score 0 | Score 1 | Score 2 | Score 3 |
| Exp. 1 | Phenytoin | 0 | 0 | 2 | 8 |
| Exp. 2 | Valproic acid | 0 | 1 | 1 | 8 |
| Exp. 3 | Cyclosporine A | 0 | 1 | 2 | 7 |
| Exp. 4 | Nifedipine | 0 | 2 | 3 | 5 |
| Exp. 5 | Diltiazem | 0 | 2 | 2 | 6 |
| Exp. 6 | Verapamil HCl | 0 | 1 | 1 | 8 |
| Exp. 7 | Amoldipine | 0 | 1 | 1 | 8 |
| Control 1 | — | 10 | 0 | 0 | 0 |

TABLE 3b
Effect on inhibiting the generation of wrinkles in hairless mice

| | | Inhibition of wrinkles (number of mice) | | | |
|---|---|---|---|---|---|
| Group | Compound (10⁻⁵ M) | Score 0 | Score 1 | Score 2 | Score 3 |
| Exp. 1 | Phenytoin | 0 | 0 | 3 | 7 |
| Exp. 2 | Valproic acid | 0 | 1 | 2 | 7 |
| Exp. 3 | Cyclosporine A | 0 | 1 | 3 | 6 |
| Exp. 4 | Nifedipine | 0 | 2 | 3 | 5 |
| Exp. 5 | Diltiazem | 0 | 2 | 2 | 6 |
| Exp. 6 | Verapamil HCl | 0 | 1 | 2 | 7 |
| Exp. 7 | Amoldipine | 0 | 1 | 3 | 6 |
| Control 1 | — | 10 | 0 | 0 | 0 |

TABLE 3c
Effect on inhibiting the generation of wrinkles in hairless mice

| | | Inhibition of wrinkles (number of mice) | | | |
|---|---|---|---|---|---|
| Group | Compound (10⁻³ M) | Score 0 | Score 1 | Score 2 | Score 3 |
| Exp. 1 | Phenytoin | 0 | 0 | 3 | 7 |
| Exp. 2 | Valproic acid | 0 | 0 | 2 | 8 |
| Exp. 3 | Cyclosporine A | 0 | 0 | 2 | 8 |
| Exp. 4 | Nifedipine | 0 | 2 | 3 | 5 |
| Exp. 5 | Diltiazem | 0 | 1 | 2 | 7 |
| Exp. 6 | Verapamil HCl | 0 | 0 | 1 | 9 |
| Exp. 7 | Amoldipine | 0 | 0 | 2 | 8 |
| Control 1 | — | 10 | 0 | 0 | 0 |

As shown in Tables 3a to 3c, the active ingredients inhibited the generation of wrinkles by a high degree in above about 80% of hairless mice. This demonstrates that active ingredients of the invention have excellent effects on inhibiting the generation of wrinkles.

### EXPERIMENTAL EXAMPLE 4

### Effect of Alleviating Wrinkles in Hairless Mice

The effects of active ingredients of the invention, that is, phenytoin, valproic acid, cyclosporinee A, nifedipine, diltiazem, verapamil HCl and amoldipine, on alleviating photo-induced wrinkles in 6-week hairless mice were investigated.

Mice were placed into 21 experimental groups and 3 control groups, with 10 rats per group. For experimental groups, mice were applied to the skin with respective active ingredients at a concentration of 10⁻⁸ to 10⁻³ M. The control groups were the mice applied with solvent only without any active ingredient. The experimental protocol is as follows. Hairless mice were radiated using a simulated sunlight at a dose of 2 MED (double Minimal Erythema Dose) 3 days a week for 10 weeks, thereby generating wrinkles. Then, respective active ingredients or the solvent only were applied at a volume of 100 μl each, twice a day for 6 weeks. Degrees of wrinkle reduction were determined. The determination was performed by visually observing the compound-applied region with naked eyes, and the region was photographed. The degrees of alleviation of wrinkles in the compound-treatment groups (experimental groups) were compared with those of the control group and were determined as one of 4 stages, that is, none (Score 0), slight (Score 1), moderate (Score 2) and high (Score 3), and the corresponding mice were counted. The data are shown in Tables 4a to 4c.

TABLE 4a
Effect of alleviating wrinkles in hairless mice

| | | Reduction of wrinkles (number of mice) | | | |
|---|---|---|---|---|---|
| Group | Compound (10⁻⁸ M) | Score 0 | Score 1 | Score 2 | Score 3 |
| Exp. 1 | Phenytoin | 0 | 1 | 2 | 7 |
| Exp. 2 | Valproic acid | 0 | 1 | 2 | 7 |
| Exp. 3 | Cyclosporine A | 0 | 2 | 3 | 5 |
| Exp. 4 | Nifedipine | 0 | 2 | 3 | 5 |
| Exp. 5 | Diltiazem | 0 | 1 | 2 | 7 |
| Exp. 6 | Verapamil HCl | 0 | 2 | 2 | 6 |
| Exp. 7 | Amoldipine | 0 | 1 | 1 | 8 |
| Control 1 | — | 9 | 1 | 0 | 0 |

TABLE 4b
Effect of alleviating wrinkles in hairless mice

| | | Reduction of wrinkles (number of mice) | | | |
|---|---|---|---|---|---|
| Group | Compound (10⁻⁵ M) | Score 0 | Score 1 | Score 2 | Score 3 |
| Exp. 1 | Phenytoin | 0 | 1 | 3 | 6 |
| Exp. 2 | Valproic acid | 0 | 1 | 2 | 7 |
| Exp. 3 | Cyclosporine A | 0 | 2 | 2 | 6 |
| Exp. 4 | Nifedipine | 0 | 2 | 2 | 6 |
| Exp. 5 | Diltiazem | 0 | 1 | 2 | 7 |
| Exp. 6 | Verapamil HCl | 0 | 2 | 1 | 7 |
| Exp. 7 | Amoldipine | 0 | 2 | 2 | 6 |
| Control 1 | — | 9 | 1 | 0 | 0 |

TABLE 4c
Effect of alleviating wrinkles in hairless mice

| | | Reduction of wrinkles (number of mice) | | | |
|---|---|---|---|---|---|
| Group | Compound (10⁻³ M) | Score 0 | Score 1 | Score 2 | Score 3 |
| Exp. 1 | Phenytoin | 0 | 0 | 3 | 7 |
| Exp. 2 | Valproic acid | 0 | 0 | 2 | 8 |
| Exp. 3 | Cyclosporine A | 0 | 2 | 2 | 6 |
| Exp. 4 | Nifedipine | 0 | 2 | 2 | 6 |
| Exp. 5 | Diltiazem | 0 | 0 | 2 | 8 |
| Exp. 6 | Verapamil HCl | 0 | 1 | 2 | 7 |
| Exp. 7 | Amoldipine | 0 | 1 | 3 | 6 |
| Control 1 | — | 8 | 2 | 0 | 0 |

As shown in Tables 4a to 4c, the active ingredients exhibited a high level of alleviation effects on the photo-induced wrinkles in above about 80% of hairless mice. This demonstrates that active ingredients of the invention have excellent effects on alleviating wrinkles.

The results from the experiments employing the active ingredients of the invention for evaluating effects of promoting collagen synthesis in fibroblasts derived from human, rats and mice demonstrate that phenytoin, valproic acid, cyclosporinee A, nifedipine, diltiazem, verapamil HCl and amoldipine with concentrations of 10⁻⁸ to 10⁻³ M each have excellent effects of promoting collagen synthesis.

A topical composition comprising an active ingredient of the invention may include any formulations applicable to skin, for example, cream, ointment, lotion, skin tonic, gel, pack, aerosol types thereof, patch and patch-type apparatus with micro needles. The compositions were especially prepared in formulations of cream, ointment and pack and applied to human skin for evaluating reduction of wrinkles. It was found that they significantly reduce wrinkle density.

Hereinafter, the present invention will be described in detail, in conjunction with examples and comparative examples. It is noted that these examples are provided only for illustrative purposes, and the present invention is not to be construed as being limited to those examples.

Preparation of Variable Formulations Comprising an Active Ingredient of the Invention

Agents topically applicable to the skin were prepared with compositions given in Tables 5 to 7, employing each active ingredient and other supplementary components according to the invention. In the invention, ointment, cream, pack, essence, skin softner, nutrient emulsion, patch and patch-type apparatus with micro needles, each topically applicable to the skin, were prepared. It is noted that though only formulations employing phenytoin and cyclosporine A as active ingredients were prepared herein, the examples are not intended to limit the formulations and active ingredients.

TABLE 5
Formulation of ointment

(unit: weight %)

| | | | | | Comp. |
|---|---|---|---|---|---|
| Component | Ex. 1 | Ex. 2 | Ex. 3 | Ex. 4 | Ex. 1 |
| Diethyl Sebacate | 8 | 8 | 8 | 8 | 8 |
| Spermaceti | 5 | 5 | 5 | 5 | 5 |
| Polyoxyethylene* | 6 | 6 | 6 | 6 | 6 |
| Sodium benzoate | typical | typical | typical | typical | typical |
| Phenytoin | 0.00001 | 0.1 | — | — | — |
| Cyclosporine A | — | — | 0.00001 | 0.1 | — |
| Total weight with | 100 | 100 | 100 | 100 | 100 |
| Vaseline added | | | | | |

*Polyoxyethylene oleic ether phosphate

TABLE 6
Formulation of cream

(unit: weight %)

| Component | Ex. 1 | Ex. 2 | Ex. 3 | Ex. 4 | Comp. Ex. 1 |
|---|---|---|---|---|---|
| Stearic acid | 15.0 | 15.0 | 15.0 | 15.0 | 15.0 |
| Setanol | 1.0 | 1.0 | 1.0 | 1.0 | 1.0 |
| Potassium | 0.7 | 0.7 | 0.7 | 0.7 | 0.7 |
| hydroxide | | | | | |
| Glycerin | 5.0 | 5.0 | 5.0 | 5.0 | 5.0 |
| Propylene | 3.0 | 3.0 | 3.0 | 3.0 | 3.0 |
| glycol | | | | | |
| Preservative | typical | typical | typical | typical | typical |
| Flavor | typical | typical | typical | typical | typical |
| Phenytoin | 0.00001 | 0.001 | — | — | — |
| Cyclosporine A | — | — | 0.0001 | 0.001 | — |
| Total | 100 | 100 | 100 | 100 | 100 |
| weight with | | | | | |
| purified | | | | | |
| water added | | | | | |

TABLE 7
Formulation of pack

(unit: weight %)

| | | | | | Comp. |
|---|---|---|---|---|---|
| Component | Ex. 1 | Ex. 2 | Ex. 3 | Ex. 4 | Ex. 1 |
| Glycerin | 5.0 | 5.0 | 5.0 | 5.0 | 5.0 |
| Propylene glycol | 4.0 | 4.0 | 4.0 | 4.0 | 4.0 |
| Polyvinyl alcohol | 15.0 | 15.0 | 15.0 | 15.0 | 15.0 |
| Ethanol | 8.0 | 8.0 | 8.0 | 8.0 | 8.0 |
| Polyoxyethylene | 1.0 | 1.0 | 1.0 | 1.0 | 1.0 |
| oleic ethyl | | | | | |
| Paraoxy methyl benzoate | 0.2 | 0.2 | 0.2 | 0.2 | 0.2 |
| Flavor | typical | typical | typical | typical | typical |
| Phenytoin | 0.1 | 0.5 | — | — | — |
| Cyclosporine A | — | — | 0.1 | 0.5 | — |
| Total weight with | 100 | 100 | 100 | 100 | 100 |
| purified water added | | | | | |

TABLE 8
Formulation of essence

(unit: weight %)

| Component | Ex. 1 | Ex. 2 | Ex. 3 | Ex. 4 | Comp. Ex. 1 |
|---|---|---|---|---|---|
| Cyclometicon | 15.0 | 15.0 | 15.0 | 15.0 | 15.0 |
| Caprilic/capric | 3.0 | 3.0 | 3.0 | 3.0 | 3.0 |
| triglyceride | | | | | |
| Mineral oil | 3.0 | 3.0 | 3.0 | 3.0 | 3.0 |
| beeswax | 1.0 | 1.0 | 1.0 | 1.0 | 1.0 |
| Cetyl dimethicone | 3.0 | 3.0 | 3.0 | 3.0 | 3.0 |
| copolyol | | | | | |
| Glycerin | 5.0 | 5.0 | 5.0 | 5.0 | 5.0 |
| Magnesium sulfate | 3.0 | 3.0 | 3.0 | 3.0 | 3.0 |
| Paraoxy benzoate | typical | typical | typical | typical | typical |
| ester | | | | | |
| Phenytoin | 0.01 | 0.05 | — | — | — |
| Cyclosporine A | — | — | 0.01 | 0.05 | — |
| Total weight with | 100 | 100 | 100 | 100 | 100 |
| purified water added | | | | | |

TABLE 9
Formulation of skin softner

(unit: weight %)

| | | | | | Comp. |
|---|---|---|---|---|---|
| Component | Ex. 1 | Ex. 2 | Ex. 3 | Ex. 4 | Ex. 1 |
| Glycerin | 2.0 | 2.0 | 2.0 | 2.0 | 2.0 |
| Hyaluronic acid | 1.0 | 1.0 | 1.0 | 1.0 | 1.0 |
| Polyoxyethylene | 0.1 | 0.1 | 0.1 | 0.1 | 0.1 |
| oleic ether | | | | | |
| Polyoxyethylene | 0.1 | 0.1 | 0.1 | 0.1 | 0.1 |
| hydrogenated castor | | | | | |
| oil | | | | | |
| Paraoxy benzoate | typical | typical | typical | typical | typical |
| ester | | | | | |
| Flavor | typical | typical | typical | typical | typical |
| Colorant | typical | typical | typical | typical | typical |
| Phenytoin | 0.0001 | 0.001 | — | — | — |
| Cyclosporine A | — | — | 0.0001 | 0.001 | — |
| Total weight with | 100 | 100 | 100 | 100 | 100 |
| purified water | | | | | |
| added | | | | | |

TABLE 10
Formulation of nutrient emulsion

(unit: weight %)

| | | | | | Comp. |
|---|---|---|---|---|---|
| Component | Ex. 1 | Ex. 2 | Ex. 3 | Ex. 4 | Ex. 1 |
| Setanol | 1.0 | 1.0 | 1.0 | 1.0 | 1.0 |
| Beeswax | 0.5 | 0.5 | 0.5 | 0.5 | 0.5 |
| Vaseline | 2.0 | 2.0 | 2.0 | 2.0 | 2.0 |
| Squalene | 6.0 | 6.0 | 6.0 | 6.0 | 6.0 |
| Ethanol | 3.0 | 3.0 | 3.0 | 3.0 | 3.0 |
| 1,3-butyleneglycol | 4.0 | 4.0 | 4.0 | 4.0 | 4.0 |
| Polysolbait 60 | 1.0 | 1.0 | 1.0 | 1.0 | 1.0 |
| Solbitan sesqi oleate | 0.3 | 0.3 | 0.3 | 0.3 | 0.3 |
| Carboxy- | 0.3 | 0.3 | 0.3 | 0.3 | 0.3 |
| vinylpolymer | | | | | |
| Triethanol amine | 0.3 | 0.3 | 0.3 | 0.3 | 0.3 |
| Paraoxy benzoate | typical | typical | typical | typical | typical |
| ester | | | | | |
| Flavor | typical | typical | typical | typical | typical |
| Colorant | typical | typical | typical | typical | typical |
| Phenytoin | 0.0001 | 0.001 | — | — | — |
| Cyclosporine A | — | — | 0.0001 | 0.001 | — |
| Total weight with | 100 | 100 | 100 | 100 | 100 |
| purified water added | | | | | |

TABLE 11
Formulation of patch

(unit: weight %)

| Component | Compound | Ex. 1 | Ex. 2 | Ex. 3 | Ex. 4 | Comp. Ex. 1 |
|---|---|---|---|---|---|---|
| Polymer | carboxymethylcellulose | 1 | 1 | 1 | 1 | 1 |
| | polyacrylic acid | 2 | 2 | 2 | 2 | 2 |
| Cross link agent | acetaldehyde | 0.1 | 0.1 | 0.1 | 0.1 | 0.1 |
| Humectant | glycerin | 30 | 30 | 30 | 30 | 30 |
| Inorganic filling agent | caolin | 0.1 | 0.1 | 0.1 | 0.1 | 0.1 |
| Preservative | paraoxy | 0.1 | 0.1 | 0.1 | 0.1 | 0.1 |
| | methyl benzoate | | | | | |
| | paraoxy | 0.05 | 0.05 | 0.05 | 0.05 | 0.05 |
| | propyl benzoate | | | | | |
| Buffer | monosodium phosphate | 0.1 | 0.1 | 0.1 | 0.1 | 0.1 |
| | sodium | 0.05 | 0.05 | 0.05 | 0.05 | 0.05 |
| | tripoly phosphate | | | | | |
| Active ingredient | phenytoin | 0.01 | 0.05 | — | — | — |
| | cyclosporine A | — | — | 0.01 | 0.05 | — |
| Total weight with | | 100 | 100 | 100 | 100 | 100 |
| purified water added | | | | | | |
| Support | | cotton | cotton | cotton | cotton | cotton |
| Protective film | | silicon | silicon | silicon | silicon | silicon |

With regard to a patch-type apparatus with micro needles, a main body of the patch apparatus, a reservoir which contains a solvent for a drug, is comprised of a polymer support for securing an entire patch type apparatus as well as preventing a drug such as phenytoin, valproic acid, cyclosporinee A, nifedipine, diltiazem, verapamil HCl and a moldipine from being permeable thereto. The solvent for a drug may be water, polyethyleneglycol, transcutol or ethanol and is contained inside the reservoir. As for the polymer support, polyethylene, polypropylene, non-woven fabric or cotton fabric are available. The drug mentioned above is dispersed in powdered form in a lower part of the reservoir. The patch-type apparatus is an instrument for administering a drug transdermally, characterized by further comprising a support for micro needles and a number of micro needles. As for a support for micro needles, it is made of a polymer gel such as celluloses, polypropylene, fluorocarbon or polycarbonate and it has a swelling property as the solvent is released after adhesion to the skin. As for micro needles, they are distributed and fixed perpendicular to the support for micro needles, and they come into contact with the skin. More particularly, 10 to 50 micro needles are attached per unit area (cm) of the support for needles and each has a channel through which a drug can pass, the channel being 1 to 1000 μm in diameter and the needles being fixed outward 0.01 to 1 mm in length. The apparatus has an adhesive layer at its lower part which has a role in adhering the apparatus to the skin, the adhesive layer being made of a material such as polyacrylate or polybutene. It should be noted that the adhesive layer has no adverse effects on skin and is not dissolved by a solvent. Further, no decrease in adhesive property by a solvent is permitted. Finally, there is a protective film attached to the adhesive layer film, which is easily removable upon using the apparatus, to prevent a drug from being leaked, and protecting an adhesive. Hereinafter, the present invention will be described in detail in conjunction with examples, not to be construed as being limited to those examples.

Preparation of Patch-Type Apparatus with Micro Needles Comprising an Active Ingredient of the Invention

### COMPARATIVE EXAMPLE 1

1 g of 3% gelatin solution was poured to a fabric with micro needles (15 needles/cm²) which were fixed perpendicular to the fabric and the resultant fabric was dried under vacuum using a lyophilizer. A comparative matrix was thus obtained.

### EXAMPLE 1

0.001% phenytoin was added to 1 g of 3% gelatin solution and homogenously dispersed therein. The solution was poured to a fabric with micro needles (15 needles/cm²) which were fixed perpendicular to the fabric and the resultant fabric was dried under vacuum using a lyophilizer. A phenytoin-dispersed matrix was thus obtained.

### EXAMPLE 2

0.001% cyclosporine A was added to 1 g of 3% gelatin solution and homogenously dispersed therein. The solution was poured to a fabric with micro needles ( 15 needles/cm²) which were fixed perpendicular to the fabric and the resultant fabric was dried under vacuum using a lyophilizer. A cyclosporine A-dispersed matrix was thus obtained.

Evaluation of Prevention and Treatment Effects on Skin Aging by a Composition Comprising an Active Ingredient of the Invention

To evaluate the effect of the formulations prepared in above comparative example and examples including the examples as set forth in Tables 5 to 11 on alleviating skin wrinkles, female subjects aged 35-60 were employed. 760 females were placed into 38 groups, 20 subjects per group. Respective examples and comparative examples were applied to the face twice per day for 3 months (in case of the packs of Table 7, they were removed 30 min after application). The degrees of alleviating wrinkles were determined by a survey and an image analysis of wrinkles after 3 months. As for the survey, the degrees of alleviation of wrinkles and increase of elasticity were determined as one of 4 stages, that is, none, slight, moderate and high, as compared with the conditions before applying respective compositions, and the corresponding subjects were counted. The data are shown in Tables 12. For the evaluation by an image analysis of wrinkles, one replica of the region right below the eye of each subject was taken using Xantopren (Bayer) before beginning the experiment. Another replica was taken in the same region immediately after finishing the experiment. The replicas were subjected to an image analysis. Wrinkle density was measured by a two dimensional analysis. The measurements were represented as decrease rates, relative to wrinkle densities before the experiment. The results are shown in Table 13.

TABLE 12
Alleviation of wrinkles in human females

| Degree of alleviation | Example | None | Slight | Moderate | High |
|---|---|---|---|---|---|
| Ointment | Ex. 1 | 1* | 3 | 4 | 12 |
| | Ex. 2 | 0 | 2 | 4 | 14 |
| | Ex. 3 | 0 | 4 | 7 | 9 |
| | Ex. 4 | 0 | 3 | 6 | 11 |
| | Comp. Ex. 1 | 17 | 3 | 0 | 0 |
| Cream | Ex. 1 | 0 | 1 | 8 | 11 |
| | Ex. 2 | 0 | 1 | 6 | 13 |
| | Ex. 3 | 0 | 2 | 7 | 11 |
| | Ex. 4 | 0 | 3 | 7 | 10 |
| | Comp. Ex. 1 | 13 | 7 | 0 | 0 |
| Pack | Ex. 1 | 0 | 0 | 9 | 11 |
| | Ex. 2 | 0 | 2 | 5 | 13 |
| | Ex. 3 | 0 | 3 | 4 | 14 |
| | Ex. 4 | 0 | 1 | 5 | 14 |
| | Comp. Ex. 1 | 15 | 5 | 0 | 0 |
| Essence | Ex. 1 | 0 | 2 | 5 | 13 |
| | Ex. 2 | 0 | 0 | 5 | 15 |
| | Ex. 3 | 0 | 3 | 6 | 11 |
| | Ex. 4 | 0 | 1 | 7 | 12 |
| | Comp. Ex. 1 | 12 | 7 | 1 | 0 |
| Skin softner | Ex. 1 | 0 | 2 | 8 | 10 |
| | Ex. 2 | 0 | 1 | 7 | 12 |
| | Ex. 3 | 0 | 3 | 7 | 10 |
| | Ex. 4 | 0 | 4 | 5 | 11 |
| | Comp. Ex. 1 | 16 | 4 | 0 | 0 |
| Nutrient emulsion | Ex. 1 | 0 | 1 | 5 | 14 |
| | Ex. 2 | 0 | 0 | 5 | 15 |
| | Ex. 3 | 0 | 1 | 7 | 12 |
| | Ex. 4 | 0 | 2 | 7 | 11 |
| | Comp. Ex. 1 | 13 | 7 | 0 | 0 |
| Patch | Ex. 1 | 0 | 1 | 4 | 15 |
| | Ex. 2 | 0 | 0 | 4 | 16 |
| | Ex. 3 | 0 | 1 | 7 | 12 |
| | Ex. 4 | 0 | 1 | 9 | 10 |
| | Comp. Ex. 1 | 15 | 5 | 0 | 0 |
| Micro-needle patch | Ex. 1 | 0 | 1 | 4 | 15 |
| | Ex. 2 | 0 | 1 | 9 | 10 |
| | Comp. Ex. 1 | 15 | 5 | 0 | 0 |

*the number of subjects counted

TABLE 13
Effect of decreasing wrinkle density in human females

| Ex- | Oint- | | | | Skin | Nutrient | |
|---|---|---|---|---|---|---|---|
| ample | ment | Cream | Pack | Essence | softner | emulsion | Patch |
| Ex. 1 | 45% | 43% | 40% | 39% | 44% | 45% | 46% |
| Ex. 2 | 44% | 41% | 38% | 37% | 42% | 43% | 48% |
| Ex. 3 | 50% | 40% | 41% | 40% | 48% | 46% | 45% |
| Ex. 4 | 48% | 50% | 44% | 39% | 45% | 42% | 44% |
| Comp. | 98% | 98% | 94% | 97% | 99% | 98% | 96% |
| Ex. 1 | | | | | | | |

As shown in Table 12, the examples according to the invention provide excellent effects of alleviating wrinkles and increasing skin elasticity. Specifically, more than 80% showed high levels of improving effects. As shown in Table 13, when the examples comprising an active ingredient of the invention were applied to the subjects, wrinkle densities were considerably decreased to about 37 to 50%, compared to that before the experiment. Also, when a patch-type administering apparatus with micro needles was applied, examples 1 and 2 exhibited significant decreases in wrinkle densities, 70% and 60% respectively, indicating that the examples are superior to the comparative example (98%) (data not shown).

The above experimental results demonstrate that when the active ingredients of the invention are topically applied to the skin in the form of cream, ointment, lotion, skin tonic, gel, pack, patch, or patch-type apparatus with micro needle, skin wrinkles generated by intrinsic or extrinsic causes are effectively alleviated.

Industrial Applicability

As apparent from the above description, the present invention provides a topical composition which comprises one or two or more selected from the group consisting of phenytoin, valproic acid, cyclosporine A, nifedipine, diltiazem, verapamil HCl and amoldipine as an active ingredient having an effect of promoting collagen synthesis, exhibiting the effects of inhibiting, alleviating and preventing skin aging, such as skin wrinkles.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A topical composition comprising at least one selected from the group consisting of phenytoin, valproic acid and cyclosporine A as an active ingredient having an effect of promoting collagen synthesis for prevention and alleviation of skin wrinkles.

2. The composition as set forth in claim 1, wherein the active ingredient is contained at an amount of 0.00001 to 30.00% by weight, relative to the total weight of the composition.

3. The composition as set forth in claim 1 or claim 2, wherein the composition is formulated in a form of cream, ointment, lotion, skin tonic, gel, pack, patch, or patch-type administering apparatus with micro needles.
